SEQUENCE LISTING (2 sequences)

SEQ ID NO: 1 - protein, 10 aa, Artificial Sequence
  Peptide antigen
GLKDRKRPSE

SEQ ID NO: 2 - protein, 10 aa, Artificial Sequence
  Peptide antigen
QVKYAGSQVD

(19) United States
(12) Patent Application Publication
(10) Pub. No.: US 2022/0033474 A1
(43) Pub. Date: Feb. 3, 2022

(54) STRUCTURE-SENSITIVE PEPTIDE ANTIGEN OF VON WILLEBRAND FACTOR

(71) Applicant: SOOCHOW UNIVERSITY, Suzhou (CN)

(72) Inventor: Wei DENG, Suzhou (CN)

(21) Appl. No.: 17/051,761

(22) PCT Filed: Oct. 11, 2019
(86) PCT No.: PCT/CN2019/110564

§ 371 Date: Aug. 18, 2021

(30) Foreign Application Priority Data
Sep. 24, 2019 (CN) 201910906491.9

(51) Int. Cl. C07K 14/755; C07K 16/36; G01N 33/86; A61K 39/00

(52) U.S. Cl. CPC C07K 14/755; C07K 16/36; G01N 2800/224; A61K 39/0005; G01N 2333/755; G01N 33/86

(57) ABSTRACT

The present invention discloses a polypeptide antigen sequence, its spatially sensitive position in von Willebrand factor (VWF), and use thereof in the production of preparations for the diagnosis and/or treatment of VWF-related blood diseases. The polypeptide antigen comprises the amino acid sequence as shown in SEQ ID NO: 1, or a biologically active fragment or a variant thereof. The spatial position of the polypeptide transits from non-solvent exposure to solvent exposure when VWF is in activation process upon exposing to blood shear stress, so as to be recognized by an antibody. The antibody that recognizes the active state of VWF prepared by using the polypeptide antigen is useful in diagnosing and treating a variety of VWF-related diseases.

### FIELD OF THE INVENTION

The present invention relates to the field of preparations recognizing von Willebrand factor (VWF), and in particular to a sequence of a structure-sensitive peptide antigen of von Willebrand factor.

### DESCRIPTION OF THE RELATED ART

VWF is a very important large multimeric protein released by vascular endothelial cells into the blood circulation, which mediates hemostasis, thrombosis, angiogenesis, and inflammation by directly sensing shear stress. VWF is one of the molecules that are mostly sensitive to genetic mutations. Approximately 6 million patients worldwide suffer from von Willebrand disease (VWD), which is one of the most common inherited hemorrhagic disorders due to mutations of the VWF gene.

The size of VWF multimer has a direct impact on the VWF reactivity, and is also high sensitive to the blood shear stress. The precise adjustment of the size and reactivity of VWF by the blood shear stress is essential for hemostasis. In the presence of a low shear stress, the VWF multimer assumes a loosely curled condensed shape due to the weak interaction between VWF monomers. Under these conditions, VWF does not bind platelets. Above the critical shear rate, the VWF polymer stretches and is amenable to a tensile force. Tension induces structural changes around A1, allowing it to bind to platelets and leading to platelet aggregation and clearance. The conformational change of VWF is directly related to its activity. Maladjustment of this conformational change will lead to a variety of high-risk thrombotic diseases such as stroke, myocardial infarction and pulmonary embolism.

The diagnostic principles of current preparations for diagnosing VWF activity are based on ristocetin aggregation. These diagnostic preparations are used under non-physiological conditions, and the diagnostic results are prone to interference by antibodies and other factors, so the reliability is poor. The existing preparations for the treatment of VWF-related blood diseases are mainly hormones, which lack targeting ability and durability.

Antibody drugs have high targeting ability. With the identification and recognition of molecular targets that cause diseases, antibody drugs have rapid development in recent years. Tumor targeting antibodies are represented by anti-PD-1/PD-L1 monoclonal antibody. The medicine has achieved very good therapeutic effects. Caplacizumab (trade name: Cablivi), the first anti-VWF antibody drug, was approved by FDA in February 2019. By blocking the binding of VWF to platelets, the drug reduces the symptoms of organ ischemia due to thrombosis, and is thus used to treat thrombotic thrombocytopenic purpura (TTP).

In the process of coagulation and thrombosis, VWF transits from an inactive state to an active state by changing its conformation, to bind to platelets and play its function. Caplacizumab binds undifferentiatedly to inactive and active VWF and leads to its clearance, thus causing the overall decline of VWF level. This decline may result in unpredictable bleeding risks, and with the consumption of VWF, Caplacizumab suffers from poor durability and has to be used at a large dosage. These shortcomings leadto the fact that the drug can only be used in the treatment of extremely small number of patients with TTP.

Therefore, to allow the anti-VWF antibody to be used in a wider range of von Willebrand diseases, it is necessary to find an antibody that can only target the activated VWF. This antibody will only recognize VWF in the active state and clear the VWF bound to the antibody by the liver, thereby reducing the risk of thrombosis while reducing the dose of medication, and prolonging its durability. In the literatures “Delimiting the autoinhibitory module of von Willebrandfactor, W Deng, K M Voos, J K Colucci, E R Legan, E A Ortlund, P Lollar, R LiJournal of Thrombosis and Haemostasis 16 (10), 2097-2105,” and “A discontinuous autoinhibitory module masks the A1 domain of von Willebrandfactor, W Deng, Y Wang, S A Druzak, J F Healey, A K Syed, P Lollar, R Li, Journal of Thrombosis and Haemostasis 15 (9), 1867-1877,” it is proposed that the activation of VWF is effected by the interaction between the short peptides flanking the A1 domain with A1. However, firstly, in these two literatures, activity studies are conducted only on isolated A1 domain. Compared with the full-length VWF composed of 2050 amino acids, the A1 domain contains only 205 amino acids. The conclusions from the study of the A1 domain have only limited conceptual reference value for understanding the structural changes of the overall VWF. Secondly, although the conclusions from these two literatures put forward the concept that a structure-sensitive region is present in the A1 domain, specific positions of structure-sensitive peptides are not given, and no idea to develop antibodies by taking use of the structural sensitivity of the A1 domain is provided. Finally, although the antibody development technology is sophisticated, it has high requirements for the choices of antigens. Even if the structural sensitivity information of the overall domain is known, there is no guarantee that desirable antigen information can be obtained.

### SUMMARY OF THE INVENTION

To solve the above technical problems, an object of the present invention is to provide a sequence of a structure-sensitive peptide antigen of von Willebrand factor (VWF). The present invention discloses a peptide antigen that is sensitive to the active VWF, and an antibody that only recognizes active VWF is prepared.

A first object of the present invention is to disclose use of a peptide antigen in the production of preparations for diagnosis and/or treatment of VWF-related blood diseases. The peptide antigen includes the amino acid sequence as shown in SEQ ID NO: 1, or a biologically active fragment or variant thereof, and the peptide antigen is derived from the A1 domain of human VWF.

Preferably, the peptide antigen is located in the loop region between the β3 fold and α2 helix in the A1 domain of human VWF.

Preferably, when VWF is in an active state, the peptide antigen is recognized by a specific antibody thereof.

Preferably, the peptide antigen is derived from human.

Preferably, the preparation is a vaccine composition, an antibody drug or a diagnostic kit.

A second object of the present invention is to provide an antibody that specifically recognizes a peptide antigen comprising the amino acid sequence as shown in SEQ ID NO: 1, or a biologically active fragment or a variant thereof.

Preferably, the antibody recognizes VWF in an active state; and the peptide antigen is derived from the A1 domain of human VWF. The peptide antigen is located in the loop region between the β3 fold and α2 helix in the A1 domain of human VWF.

A third object of the present invention is to provide a diagnostic preparation for determining whether a peptide antigen having the amino acid sequence as shown in SEQ ID NO: 1 is present in a sample, where the diagnostic preparation comprises the antibody as described above in the present invention.

A fourth object of the present invention is to provide a therapeutic preparation for recognizing and inhibiting VWF in an active state, where the therapeutic preparation comprises the antibody as described above in the present invention.

By virtue of the above technical solution, the present invention has the following advantages.

The present invention relates to a peptide antigen sequence, its spatially sensitive position in VWF, and use thereof in the production of preparations for the diagnosis and/or treatment of VWF-related blood diseases. The spatial position of the peptide antigen is protected by the short peptide regions flanking the A1 domain when VWF is in an inactive state, and an antibody cannot recognize it. When VWF is in an active state, it transits from non-solvent exposure to solvent exposure, and can then be recognized by an antibody. The antibody that recognizes the active VWF prepared by using the peptide antigen can be used to diagnose and treat a variety of VWF-related diseases. The antibody only recognizes and inhibits active VWF.

The above description is only a summary of the technical solutions of the present invention. To make the technical means of the present invention clearer and implementable in accordance with the disclosure of the specification, the preferred embodiments of the present invention will be described in detail with reference to the accompanying drawings.

### BRIEF DESCRIPTION OF THE DRAWINGS

FIG. 1 shows the test results of the monoclonal antibody prepared in the present invention for the recognition of active and inactive VWF;

FIG. 2 illustrates the spatial position of the peptide antigens in VWF; and

FIG. 3 shows the test results of the monoclonal antibody in the comparative example for the recognition of active and inactive VWF.

### DETAILED DESCRIPTION OF THE PREFERRED EMBODIMENTS

The specific embodiments of the present invention will be described in further detail with reference to the accompanying drawings and specific embodiments. The following embodiments are intended to illustrate the present invention, instead of limiting the scope of the present invention.

### Example 1

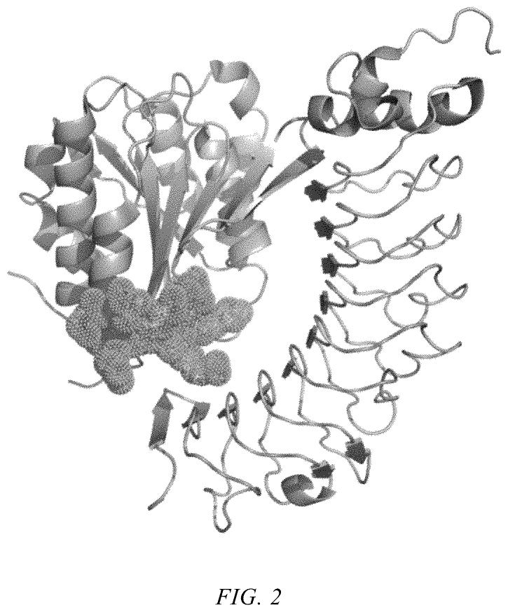

VWF consists of 2050 amino acids which are divided into 14 domains. The A1 domain is related to the coagulation function, and the A1 domain has 205 amino acids. The positions of these amino acids in the structure of the protein are known. From a region in the A1 domain of human VWF that interacts with the short peptides flanking the A1 domain, a 10-amino acid long peptide was selected as the antigen (GLKDRKRPSE, SEQ ID NO: 1). The peptide antigen is located in the loop region between the β3 fold and α2 helix in the A1 domain of human VWF (FIG. 2). A murine monoclonal antibody was prepared by a standard antibody preparation method using the peptide antigen as follows:

(1) Animal immunization: Mice were immunized with the peptide antigen of the above sequence to allow the mice produce B lymphocytes.

(2) Cell fusion: Syngeneic myeloma cells were mixed with mouse spleen cells. In the presence of polyethylene glycol, the lymphocytes and myeloma cells were fused to form hybridoma cells.

(3) Selective culture: The fused hybridoma cells were screened with HAT selective medium.

(4) Screening and clonal expansion of positive clones:

Positive hybridoma cells that can produce the required monoclonal antibodies were screened by an immunofluorescence method (ELISA), followed by clonal expansion.

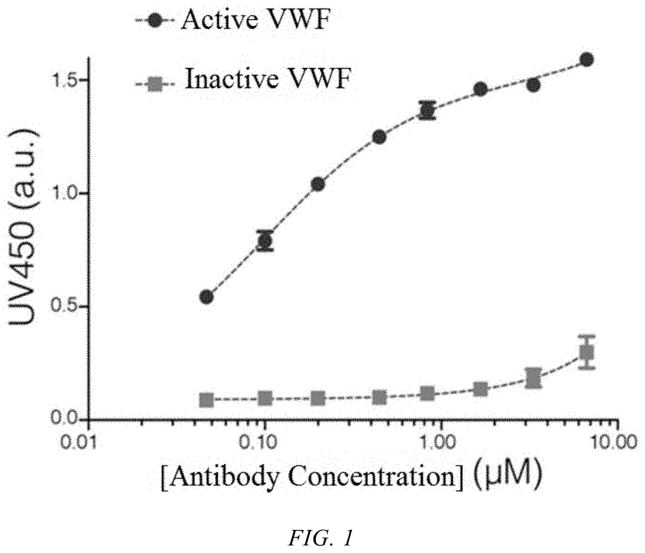

FIG. 1 shows the test results of various concentrations of the monoclonal antibody prepared in the present invention for the recognition of active and inactive VWF. The results show that as the antibody concentration increases, the UV absorption of active VWF at 450 nm is enhanced, and the UV absorption of inactive VWF at 450 nm is basically unchanged, indicating that the monoclonal antibody prepared from the peptide antigen of the present invention can specifically recognize active VWF. This is because the peptide antigen is high sensitive to the active state of VWF. VWF is protected by the short peptide regions flanking the A1 domain when it is in an inactive state, and the monoclonal antibody cannot recognize it. When VWF is activated, the peptide antigen region is exposed, and the monoclonal antibody can recognize it.

The antibody can be used to further prepare antibody drugs, which can recognize only a few active proteins, and thus will not cause the overall protein level to drop, so the risk of bleeding is very low; also, the protein will not be consumed in a large amount, and the durability is good and thus the dosage is low.

### COMPARATIVE EXAMPLES

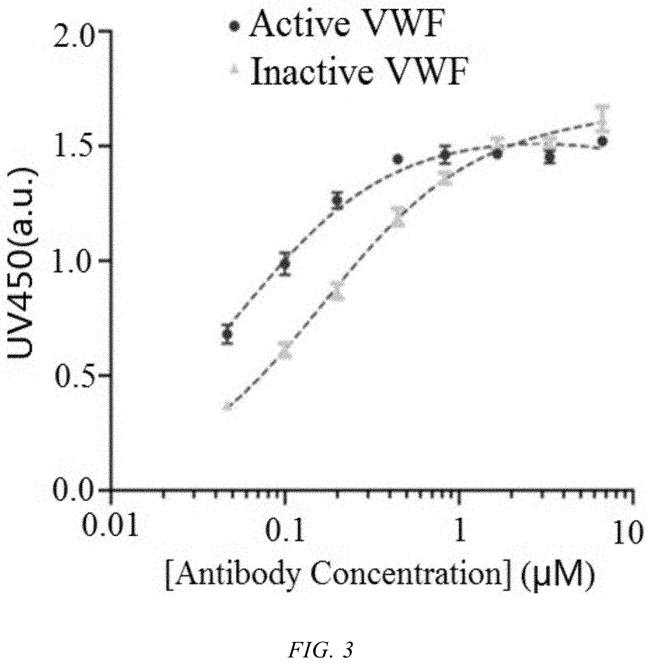

Detection of the active state of VWF with the monoclonal antibody produced by using the peptide antigen pVWF 1346-1355: QVKYAGSQVD (SEQ ID NO: 2) shows that, the antibody has a similar binding to the active and inactive states of VWF (FIG. 3), indicating that the antibody is not sensitive to the active state of VWF.

While preferred embodiments of the present invention have been described above, the present invention is not limited thereto. It should be appreciated that various improvements and variations can be made by those skilled in the art without departing from the technical principles of the present invention, which are also contemplated to be within the scope of the present invention.

1. Use of a peptide antigen in the production of preparations for diagnosis and/or treatment of blood diseases associated with von Willebrand factor (VWF), wherein the peptide antigen comprises the amino acid sequence as shown in SEQ ID NO: 1, or a biologically active fragment or variant thereof, and the peptide antigen is derived from the A1 domain of human VWF.

2. The use according to claim 1, wherein the peptide antigen is located in the loop region between the β3 fold and α2 helix in the A1 domain of VWF.

3. The use according to claim 1, wherein when VWF is in an active state, the peptide antigen is recognized by a specific antibody thereof.

4. The use according to claim 1, wherein the peptide antigen is derived from human.

5. The use according to claim 1, wherein the preparation is a vaccine composition, an antibody drug or a diagnostic kit.

6. An antibody, wherein the antibody specifically recognizes a peptide antigen comprising the amino acid sequence as shown in SEQ ID NO: 1, or a biologically active fragment or a variant thereof.

7. The antibody according to claim 6, wherein the antibody recognizes VWF in an active state; and the peptide antigen is derived from the A1 domain of human VWF.

8. A diagnostic preparation, for determining whether a peptide antigen having the amino acid sequence as shown in SEQ ID NO: 1 is present in a sample, comprising the antibody according to claim 6.

9. A therapeutic preparation, for recognizing and inhibiting VWF in an active state, comprising the antibody according to claim 1.
